(19) United States
(12) Patent Application Publication
(10) Pub. No.: US 2005/0148043 A1
(43) Pub. Date: Jul. 7, 2005

(54) Biosensor for purines

(76) Inventor: Nicholas Dale, Coventry (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/510,932

(22) PCT Filed: Apr. 3, 2003
(86) PCT No.: PCT/GB03/01467

(30) Foreign Application Priority Data
Apr. 9, 2002 (GB) 0208153.7

(51) Int. Cl. C12Q 1/32; C12M 1/34

(52) U.S. Cl. 435/26; 435/287.1

(57) ABSTRACT

Biosensors which may be used, for example, for detecting and monitoring purines such as adenosine, are disclosed. The Biosensors comprise: (i) a substrate comprising platinum or a platinum alloy; (ii) a first layer formed on the substrate, the first layer comprising a sugar-derivative of a pyrrole; and (iii) a second layer formed on the first layer, the second layer comprising an amphiphilic pyrrole and, within the second layer, one or more enzymes. Preferably, the sugar-derivative of a pyrrole is a location-amide pyrrole. Methods of producing such biosensors are also included within the scope of the claimed invention.

The application relates to biosensors, for example for detecting and monitoring purines such as adenosine, and to methods for producing such biosensors.

Since the early work by Clark and Lyons (Clark and Lyons, 1962) biosensors have evolved continuously to become a powerful analytical tool in many fields. Immobilization of active enzymes central to the operation of biosensors has been achieved in many different ways (Cass, 1990). However entrapment of enzymes in electropolymer matrices (Bartlett and Cooper, 1993) has the particular advantage of producing biosensors with very small and custom shaped sensing elements. These types of micro-biosensors are ideal for detecting neurotransmitter release in the central nervous system as they are minimally invasive.

Cosnier and coworkers have developed pyrrole derivatives that are suitable for entrapping enzymes on microelectrodes. They have used these methods to develop a variety of biosensors including sensors sensitive to glutamate (Poitry et al., 1997) and dopamine (Cosnier et al., 1997). Indeed, biosensors using biotinised derivatives are shown in U.S. Pat. No. 6,197,881B in the name of Cosnier. Although these are important signaling agents in the nervous system, the polymer films on these sensors were insufficiently robust to survive implantation into neural tissue (Poitry et al., 1997) and the sensors had mixed success at detecting release from biological tissue (Poitry et al., 1997; Cosnier et al., 1997). Nevertheless, the methods developed by Cosnier are of potentially very general utility and we have adapted them to construct robust and sensitive microelectrode biosensors that permit, spatially localized and fast detection of purine release from the nervous system. The purines, ATP and adenosine, perform extremely important signaling functions in both the peripheral and central nervous system. Peripherally, they are involved in the control of smooth muscle contraction and are powerful vasodilators (McMillan et al., 1999). Centrally, their diverse roles include regulation of spinal pain pathways (Sawynok, 1998), neuroprotection during ischaemia (Dale et al., 2000), control of transmitter release (Brundege and Dunwiddie, 1997), regulation of spinal motor pattern generation (Dale, 1998; Dale and Gilday, 1996) and induction of sleep (Porkka-Heiskanen, 1999). Adenosine, in particular, does not conform to the conventional paradigm of chemical neurotransmission in the nervous system. Instead of being released directly like most transmitters, adenosine is usually produced in the extracellular space from previously released ATP through the actions of special enzymes collectively known as the ectonucleotidases (Zimmermann and Braun, 1999). The production of adenosine—its spatial domains and kinetics of accumulation—are central to its function in the nervous system and can therefore be expected to differ considerably from those of conventional neurotransmitters. HPLC analysis of collected superfusate has been used to study adenosine release, however this method has very limited time and spatial resolution (Pedata et al., 1993). New methods for directly measuring adenosine production would thus be of great value in understanding its contribution to neural functions.

Recently, detection of adenosine produced during physiological activity has been achieved with an 3-enzyme biosensor (mark-1) (Dale, 1998) that utilizes a microdialysis electrode (250 mm diameter) to trap the required enzymes behind a semi-permeable membrane. This sensor is the subject of WO 99/07877 and is sensitive and has successfully detected release of adenosine from Xenopus embryo spinal cord (Dale, 1998) during motor activity, and mammalian hippocampus during hypoxia (Dale et al., 2000; Pearson et al., 2001). However it is too large to implant into nervous tissue without causing considerable damage and subsequent tissue reaction that may confound and invalidate physiological measurements. Furthermore the large size of the sensing assembly (500 μm when used in its finally constructed format) introduces diffusional delays which slows sensor responsiveness. To increase the range of applications for this measurement technology and to enable resolution of fast production of adenosine and related purines much smaller and faster responding biosensors are thus required.

The production of a new adenosine micro-biosensor that exhibits both more rapid responses and higher sensitivity is discussed herein. This new sensor still works on the same enzyme cascade principle as the former, but now the enzymes are immobilized on a Pt electrode using a derivatized pyrrole polymer. The inventors have improved on the methods of Cosnier (Cosnier et al., 1998) to produce the derivatized pyrroles and entrap enzymes into polymer matrices. Cosnier used glucose oxidase and polyphenol oxidase sensors with enzymes immunobilised in polymer. They used a 5 mm diameter glassy carbon disk polished with diamond paste as the working electrode.

Cosnier (1997) discusses the production of platinum electrodes having a coating of an amphiphilic pyrrole derivative and enzyme. This type of sensor is stated by the authors of the paper to be problematical. Bringing the sensor into contact with biological preparations frequently caused a partial or complete loss of sensitivity. This was thought to be due to the detachment of the polymer from the platinum surface.

The inventors have now identified an improved electrode having improved resilience. They found that coating the surface of a platinum electrode with a layer of a sugar derivatised pyrrole polymer, such as pyrrole lactobionamide, prior to coating with a layer of enzyme-containing amphiphilic pyrrole, improves the resilience of the electrode. This allows very small sensors, as small as 25 μm in diameter, to be produced, which show linear responses across a wide range of substrate concentrations.

These sensors preferably contain xanthine oxidase. The use of such an enzyme in, e.g. biosensors, is known.

EP 0537761A2 discloses a biosensor comprising a reaction layer having an oxidoreductase, such as xanthine oxidase. The reaction layer comprising an electron acceptor, such as potassium ferricyanide, p-benzoquinone, phenazinemethosulfate, methylene blue and ferrocene. The reaction layer may also comprise a hydrophilic polymer. Such biosensors are suggested for use as saccharide biosensors.

EP 0909952A2 discloses similar biosensors including a counterelectrode containing a reductant of a redox compound or a metal permitting electrolytic oxidation.

The first aspect of the invention provides a biosensor comprising:
(i) a substrate comprising a platinum or a platinum alloy;
(ii) a first layer formed on a substrate, the first layer comprising a sugar-derivative of a pyrrole; and
(iii) a second layer formed on the first layer, the second layer comprising an amphiphilic pyrrole, and within the second layer one or more enzymes.

By sugar-derivative of a pyrrole we mean that the pyrrole contains attached to it one or more sugar groups. Sugars include water-soluble carbohydrates.

The sugar groups may be attached via a linkage group such as an alkyl chain or a polyethyleneglycol (PEG) chain.

Preferably the sugar derivative comprises a general formula:

Pyrrole —R¹-Rⁿ

where:

R¹ is a straight or branched, substituted or non-substituted alkyl containing 5 to 18 carbon atoms. Preferably the alkyl contains 8, 12 or 16 carbon atoms. Alternatively, R¹ may also be (CH₂CH₂O)ₙ, where n=2 to 6, especially 2 or 4.
Rⁿ is a sugar, for example lactobionamide, glucuronamide or gluconamide.

Preferably the pyrrole is attached to lactobionamidooctane.

By amphiphilic, we mean that the pyrrole comprises at least one part of the molecule which is a polar or ionic group and a second part with a hydrocarbon group. The polar or ionic group tends to have an affinity for water, whereas the hydrocarbon group tends to have an aversion to water. Preferably the amphiphilic pyrrole comprises a tertiary amine group, such as a trimethylammonium group or a triethyl ammonium group.

Preferably the amphiphilic pyrrole has a formula:
Pyrrole-R^{III}-R^{IV}
where:
R^{III} is a straight or branched chain, substituted or non-substituted alkyl containing 5 to 18, especially 12 or 16 carbons.
R^{IV} is —N(CH₂CH₂)₃, —N(CH₃)₃, ferrocene or an osmium metal complex. One or more counterions, such as tetrafluoroborate may also be present.

Most preferably, the amphiphilic pyrrole is (12-pyrrol-1-yldodecyl)triethylammonium tetrafluoroborate.

The arrangement of the first and second layer has been found by the inventors to improve the resilience of the biosensor and to allow the biosensor to be used, for example, in biological systems, or indeed as a sensor for home-use, where gentle handling of the biosensor is unlikely. The latter sensors may be larger to enable, for example, a sample of blood or saliva to be contacted with the sensor. This allows home testing of disease markers or markers of dietary quality.

The inventors have also found that, unlike the systems used by Cosnier, the addition of a third layer comprising a sugar-derivative of a pyrrole with an alkyl linkage group can result in a lower sensitivity of the sensor. Accordingly, preferably the biosensor does not comprise a third layer consisting of a sugar-derivative linked by an alkyl chain to the pyrrole, on top of the second layer.

The inventors have realised that this problem may be overcome by linking the sugar to the pyrrole group by a polyethylene glycol (PEG) chain, which increases the hydrophillic nature of the polymer. Preferably the sensor comprises a third, outer layer, comprising a layer of general formula:
Pyrrole-R^{V}-R^{II}
where:
R^{V} is a PEG chain of general formula —(CH₂CH₂O)ₙ, where n=2 to 6, especially 2 or 4.
R^{II} is as defined above.

This may be produced using techniques known in the art. The additional layer provides a protective barrier and can prevent unwanted species entering the sensor or prevent protein contamination.

Preferably the substrate is not etched prior to coating with polymer. This has been found by the Inventors to improve the resilience of the biosensor.

Preferably the substrate is platinum or a platinum-iridium alloy, such as containing a 90:10 ratio of platinum:iridium (weight:weight).

The biosensor may comprise to or more different enzymes within the second layer. The inventors have found that building up the enzymes as separate sub-layers within the layer of amphiphilic pyrrole improves the sensitivity of the biosensor. Furthermore, several different layers of each enzyme can be built up within the layer of amphiphilic pyrrole.

Preferably, one of the enzymes is an oxidoreductase enzyme, such as xanthine oxidase. The biosensor may additionally comprise nucleoside phosphorylase and may additionally comprise adenosine deaminase.

Adenosine deaminase converts purines, such as adenosine, into inosine. Inosine, in turn, may be converted into hypoxanthine by nucleoside phosphorylase.

Finally, in the chain hypoxanthine is converted into uric acid and hydrogen peroxide by xanthine oxidase. It is the hydrogen peroxide that is detected by the platinum substrate.

Using all three enzymes allows the detection of purines, such as adenosine to be detected, as well as inosine, hypoxanthine and xanthine. Using only nucleoside phosphorylase and xanthine oxidase, inosine, hypoxanthine and xanthine may be detected. Alternatively, using only xanthine oxidase, hypoxanthine may be detected.

Preferably, an excess of xanthine oxidase compared with nucleoside phosphorylase is used. Preferably approximately equal amounts of nucleoside phosphorylase and adenosine deaminase are used.

Most preferably the ratio of adenosine deaminase:nucleoside phosphorylase:xanthine oxidase is approximately 1:1:5, based on units of activity. This ratio has been found to be the optimal ratio for this sort of electrode.

Other enzymes, such as glucose oxidase or glutamate oxidase may also be used.

Preferably the enzymes are deposited as separate sub-layers within the second layer, for example with xanthine oxidase deposited further away from the substrate than the nucleoside phosphorylase. If adenosine deaminase is present, this is deposited closer to the substrate than the nucleoside phosphorylase. Having the different enzymes in this order has been proved to give a sensor of greater sensitivity.

Preferably, the sensor comprises several layers of the enzymes.

The sensor may be used with a reference electrode, such as a silver/silver chloride reference electrode.

A further aspect of the invention provides a kit for detecting the presence and/or concentration of a substance comprising a biosensor according to the invention. The kit may comprise means for recording a current from the biosensor in comparison with a reference electrode and may also comprise means for converting the current into an indication of the presence and/or concentration of a substance. The substance may be one or more purines such as adenosine. The substance may also be xanthine and/or inosine.

Preferably the size of the electrode is less than 25 μM in diameter and may be 300 μM-2 mm long.

Alternatively, the biosensor may be fabricated into a larger biosensor for home use to enable substances to be monitored, for example in the saliva, blood or urine of a patient.

Although purine biosensors are exemplified here, use of other enzymes and enzyme cascades, for example of the sort known in the art in prior art biosensors, may be used.

A further aspect of the invention provides a method of producing a biosensor according to the invention, comprising the steps of providing a substrate comprising a platinum or a platinum alloy; depositing a first layer comprising a sugar derivative of a pyrrole; and depositing a second layer, the second layer comprising an amphiphilic pyrrole and, within the second layer, one or more enzymes.

Preferably, the second layer comprises two or more different enzymes, each enzyme being deposited sequentially as one or more separate sub-layers to form the second layer.

Preferably, the first layer is deposited in a solution comprising acetonitrile as a solvent. Preferably, the solution also contains lithium perchlorate (LiClO₄).

Preferably, the second layer comprises the amphiphilic pyrrole is also deposited in the presence of acetonitrile.

Uses of the biosensor to detect xanthine, and/or inosine, and/or one or more purines such as adenosine are also included within the scope of the invention. A method of detecting the amount of a substance within a tissue or a bodily fluid, comprising exposing a biosensor according to the invention to a sample of the tissue in vivo or in vitro, and detecting an electrical current produced by the biosensor is also provided. Preferably, the tissue is blood, brain, rough or smooth muscle or cardiac tissue. The fluid may be saliva or urine.

The invention will now be described by way of example only, with reference to the following figures:

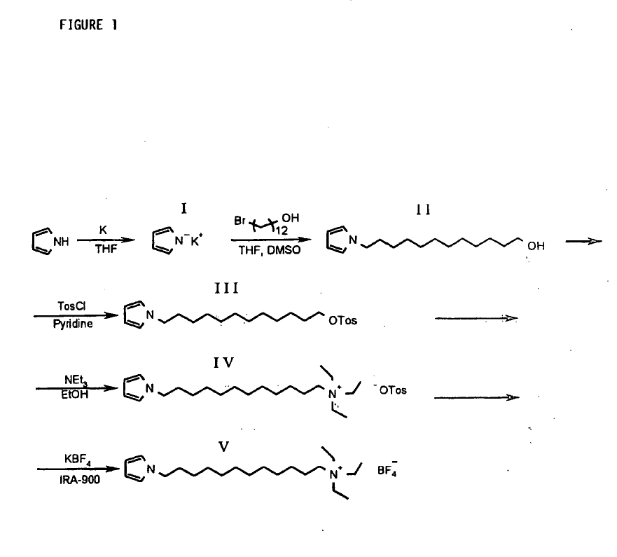

FIG. 1 shows the reaction scheme for the production of 12-pyrrol-1-yldodecyl E.=“MSJOB 3”

in contact with cord records a smaller and more delayed signal suggesting a ventral origin for adenosine production. C) Slow sensor signal abolished by coformycin (blocks adenosine deaminase) leaving transient initial increase unaffected (arrow). This demonstrates that sensor is detecting adenosine specifically. D) Null sensor (no enzymes) shows only a small baseline shift during fictive swimming once again demonstrating that signal recorded by sensor depends upon presence of enzymes.

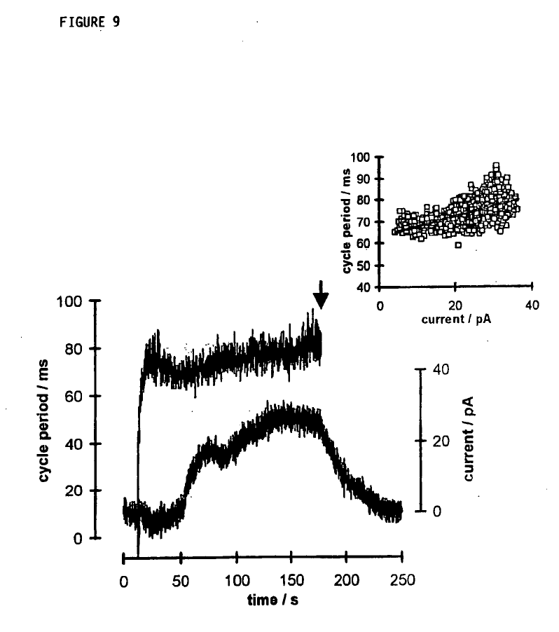

FIG. 9. Superior temporal resolution of the sensor demonstrates that accumulation of adenosine release precedes the lengthening of the cycle period of swimming. Plot of sensor current (bottom trace, right hand axis) and cycle period (top trace, left hand axis) versus time. Swimming terminates at arrow, hence the absence of cycle period measurements after this point, and the slow return of adenosine levels to the baseline. The inset shows that the lengthening of cycle period correlates with increase in sensor current. The solid line is the linear regression fit and has a slope of 0.09 ms.pA⁻¹ (equivalent to an increase in cycle period of 1 ms per 22 nM change in adenosine).

Experimental Section

Reagents and Solutions

The three enzymes, adenosine deaminase (AD), nucleoside phosphorylase (PNP) and xanthine oxidase (XO), were purchased from Sigma. Physiological saline containing 115 mM NaCl, 2.4 mM NaHCO₃, 10 mM HEPES, 3 mM KCl, 1 mM MgCl₂, 2 mM CaCl₂, 1 mM NaH₂PO₄, 1 mM Na₂HPO₄, at pH 7.4 was used in all experiments to test and use the sensor. The pyrrole derivative monomers were synthesised by adapting the previous work of Cosnier (Cosnier et al., 1998) and Deronzier (Deronzier and Moutet, 1989). We have modified their synthetic schemes so that the target molecules are easier and quicker to reach. The synthesis of monomer 1 ((12-Pyrrol-1-yldodecyl)triethylammonium Tetrafluoroborate) and monomer 2 (8-Pyrrol-1-lactobionamidooctane) are summarized in schemes 1 and 2, respectively.

### Synthesis of Amphiphillic Pyrrole (Monomer 1): (12-Pyrrol-1-yldodecyl)triethylammonium Tetrafluoroborate (FIG. 1)

Briefly, potassium metal (I molar eq.) was added in small pieces to a solution of pyrrole (0.97 molar eq.) in dry THF, the mixture was stirred under nitrogen for 12 hours. After filtration the yellow solid was washed with cold THF and vacuum dried. Pyrrolyl potassium (I) (I molar eq.) and 12-bromododecanol (0.5 molar eq.) were refluxed for 30 min in a mixture of dry THF and dry DMSO (4:1). The resulting solution was diluted with water and extracted with DCM. The organic phase was dried over Na2SO4 and rotary evaporated. The desired product was purified by chromatography on a silica column eluted with a 1:1 heptane-diethylether mixture. A solution of 12-pyrrol-1-yldodecan-1-ol (II) (1 molar eq.) and tosyl chloride (1 molar eq.) in anhydrous pyridine was stirred at 5° C. for 12 hours. The mixture was poured into water and extracted with diethylether. The organic phase was washed four times with 5% HCl aqueous solution and once with water; after drying over Na2SO4 the solvent was removed under reduced pressure. The desired product was purified by chromatography on a silica column eluted with a 1:1 heptane-diethylether mixture. 12-Pyrrol-1-yldodecyl p-Toluenesulfonate (III) (1 molar eq.) was refluxed for 24 hours in dry ethanol in the presence of triethylamine (3 molar eq.). After vacuum evaporation of the solvent and excess amine the product was purified by chromatography on a silica column eluted with a 9:1 CH3CN-H2O mixture. (12-Pyrrol-1-yldodecyl)triethylammonium Tosylate (IV) was dissolved in 1:1 water-methanol mixture (˜10 ml/100 mg), the solution was then stirred with anion-exchange resin (Amberlite IRA 900-Cl) in BF4- for 1 hour. After filtration this process was repeated four times. Evaporation of the solvent by freeze-drying gave the desired product as white powder. Final purification by chromatography on a silica column eluted with a 9:1 CH3CN—H2O mixture lead to the pure desired product (V).

¹H NMR (CDCl₃) d (ppm) 1.3 (m, 25H, H-3, H-7), 1.60 (m, 2 H, H-4), 1.70 (t, J=17 Hz, 2 H, H-2), 3.10 (q, J=3.5 Hz, 2H, H-5), 3.3 (m, 6H, H-6), 3.85 (t, J=7.6 Hz, 2H, H-1), 6.10 (t, J=1.5 Hz, 2H, H-a), 6.6 (t, J=1.5 Hz, 2H, H-b); MS (TOF) m/z, M⁺, 335.

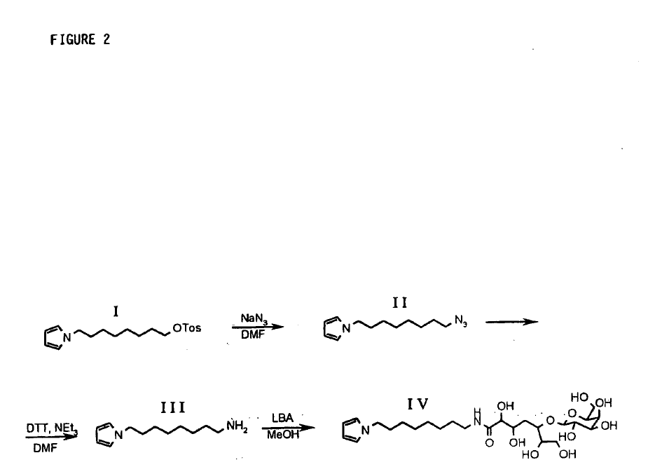

### Synthesis of the Lactobionamide Pyrrole (Monomer 2): 8-Pyrrol-1-lactobionamidooctane (FIG. 2).

In brief, 8-Pyrrol-1-yloctacyl p-Toluenesulfonate (I) (1 molar.) was refluxed in dry DMF with sodium azide (5 molar.) until the reaction was complete. After removal of the solvent under reduced pressure, the residue was dissolved in Et2O and the insoluble salts were filtered off. After vacuum evaporation of the solvent the azide intermediate (II) was obtained as an orange oil. This product was then treated with an excess of Dithiothreitol (5 molar.) in DMF and triethylamine (5 molar.) for 30 min at R.T. The resulting mixture was then poured into water and extracted with Et2O. After drying the solvent over Na2SO4 and rotatory evaporation the desired product was obtained as a yellow oil. 8-Pyrrol-laminooctane (III) (1 molar.) was added to a solution of lactobionic acid (1 molar.) in methanol, the mixture was refluxed for 24 hours. The solvent was evaporated under vacuum and the product was obtained as a pale yellow powder (IV).

¹H NMR (CDCl₃) d (ppm) 1.25 (m, 12H, H-2), 1.45 (m, 2H, H-3), 1.7 (m, 2H, H-1), 3-5.5 (m, 23H, H-5), 6.1 (m, 2H, H-a), 6.6 (m, 2H, H-b), 7.8 (t,1H, 1-1-4); MS (TOF) m/z, M+Na, 557.

Solubilization of Monomers

Monomer 1 solution was made by mixing 5 mg of monomer with 1 ml of water and vigorously stirring with a vortex, the mixture was then sonicated for 5 minutes, after adding 10% (v/v) CH₃CN and mixing again a white suspension was obtained. Electropolymerization of monomer 2 was performed in 0.1M LiClO₄ deacrated aqueous solution with 10% (v/v) CH₃CN.

Apparatus

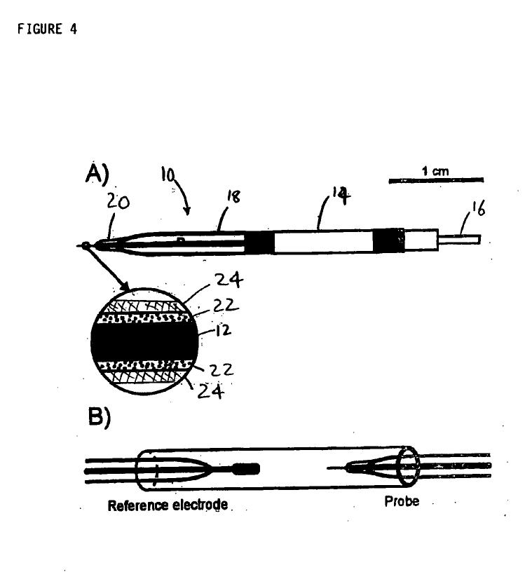

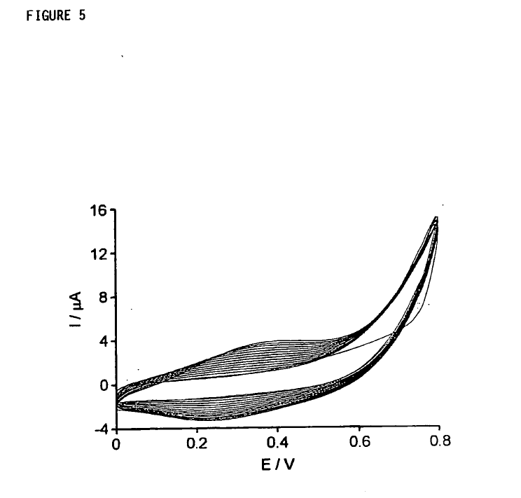

A potentiostat (Model AEW-2) from Sycopel was used to electrochemically deposit the different polymers and test the sensor. The sensor was used in vivo with a World Precision Instruments Micro C potentiostat interfaced to a PC by an A to D converter board (Data Translation). In all cases an Ag/AgCl was used as reference electrode; no counter electrode was needed due to the small size of the working electrode. The electrochemical cell for deposition consisted of a capillary of 1.5 mm diameter and 2 cm length (FIG. 4B).

Sensor Fabrication

Assembly and Cleaning (See FIG. 4)

The microelectrode (10) was assembled by soldering 2 cm of sensing wire (12) to a copper wire (14) with a terminating pin (16). Initially 250 mm pure Pt wire was used, which was subsequently etched to the desired final diameter. However pure Pt wire is very soft, limiting the smallest usable diameter to around 50 mm. To make sensors of even smaller diameter Pt/Ir wire (90/10 from Goodfellow Metals) in diameters ranging from 25 to 100 mm. was also used. This Pt/Ir wire is much stiffer, yet can still be used to make highly sensitive electrochemical sensors.

All but the final 2 mm of the Pt wire was protected by a pulled glass capillary (18) that was fused by heat to the wire. The central part of the assembly was insulated with heat shrink tubing. The exposed tip of Pt was then etched under visual inspection by 1.2 V AC electrolysis in 2M NaCl using a spiral Pt coil as counter electrode (Slevin et al., 1999). The final diameter ranged from 25 mm to 100 mm depending on the extent of etching. Note that the 25 mm Pt/Ir wire was sufficiently small to be used without further etching. In addition we found that excessive etching of the Pt/Ir wire produced a surface unfavorable for polymer deposition.

The exposed electrode was then coated with Sylgard (resin 184, Dow-Corning) (20) to leave a final, length of exposed Pt. This length was varied to suit the experimental requirements and we have constructed sensors that range in length from 300 mm to 2 min. Careful surface preparation of the Pt electrode was crucial to the ability to deposit the pyrrole polymer and to the overall sensitivity of the sensor. Without careful cleaning of the Pt surface, the polymer layers would not grow sufficiently well to entrap the enzymes efficiently. The Pt electrode was therefore cleaned by cycling in 0.1 M H₂SO₄ from −100 mV to 1000 mV (versus Ag/AgCl reference, scan rate 100 mV/s) for 15 times. Before polymer deposition the electrode was held at 1000 mV for 1 minute.

Deposition of Polymers

All sensors involved a first layer of LBA derivative polymer (22) formed by cycling the electrode 15 times from 0 to 800 mV, scan rate 100 mV/s in a de-aerated solution of monomer 2 (10 mM) in 0.1 M LiClO₄, 10% CH₃CN (FIG. 2). After this procedure the exposed Pt appeared black in colour. The unbound monomer was removed by washing the electrode in stirred dH₂O for 5 min. To entrap the enzymes the electrode was then immersed in a solution of monomer 1 (5 mg/ml, 10% CH₃CN) containing the desired enzyme, and the potential held to 760 mV for 10 minutes (24). To save monomer and enzymes, the polymer deposition was carried in a mini-chamber of 10 mL volume consisting of a short capillary glass tube (FIG. 4B).

To make an adenosine sensor three layers were deposited from solutions of monomer 1, firstly 1 U of AD in 10 ml was used, followed by 1 U of PNP in 10 ml and then 5 U of XO also in 10 ml. This procedure could be optionally repeated to give a sensor of greater sensitivity. Before testing the sensor was stirred in phosphate buffer for 5 minutes to wash unbound monomer and enzyme. Null sensors were made by depositing the same first layer of LBA polymer followed by a second layer of amphiphilic polymer, deposited for 5 min. at 760 mV from a solution of monomer 1 with no enzymes.

Sensor Characterization and Use

The sensors were tested in a 250 ml chamber continually superfused by an apparatus that permitted rapid solution changes. The sensor was polarised to 500 mV for all measurements.

To detect adenosine release from spinal cord of the Xenopus embryo during fictive swimming, stage 37/38 Xenopus embryos were prepared for recording by means of well-established techniques (Dale, 1998; Dale and Gilday, 1996). The sensor, mounted on a micromanipulator, was lowered under visual control (through a stereomicroscope) until gentle contact was made with the spinal cord. The sensor was polarized to 500 mV and after the sensor current had stabilized, the embryo was stimulated to produce swimming episodes, which were monitored from ventral root activity.

The cycle period of motor activity (which progressively increases throughout the episode of swimming from around 40 to 100 ms) was measured by a threshold crossing method, which identified the peaks of the ventral root bursts. The cycle period was calculated as the time difference between the peaks of successive ventral root bursts.

Results and Discussion

Sensor Characteristics

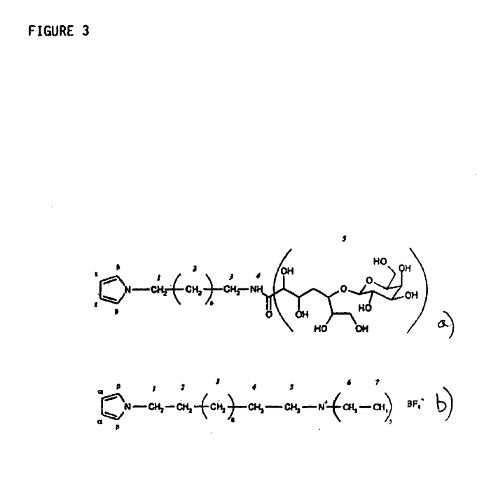

As each of the enzymes used in the sensor is susceptible to product inhibition the ideal design has to ensure that each step in the cascade removes the product of the previous step. In the earlier mark-1 sensor a ratio 100:2:1 units (XO:PNP:AD) was found to produce linear and sensitive responses (Dale, 1998). Because the sensor responds to external adenosine, inosine and xanthine the specific load of each of the 3 enzymes can be checked by comparing the responses for the 3 metabolites. In the polymer sensor the optimal ratio of enzymes in the monomer solutions was found to be 5:1:1. This produced a signal ratio (xanthine:inosine:adenosine) of 7.5±1.2:1.1±0.02:1 (mean±sem, n=8, FIG. 3A), ensuring that the product of each enzyme is cleared by the next thus avoiding product inhibition. The big difference between the enzyme ratio used for the mark-1 and the mark-2 sensors suggests that the entrapment efficiency of the enzymes varies from each other. Presumably the different sizes, charge distribution and hydrophobicity of each protein will make it interact differently with the two monomers. In contrast with Cosnier's findings (Coche-Guerente et al., 1995) xanthine oxidase seems to be trapped more easily than the two smaller enzymes.

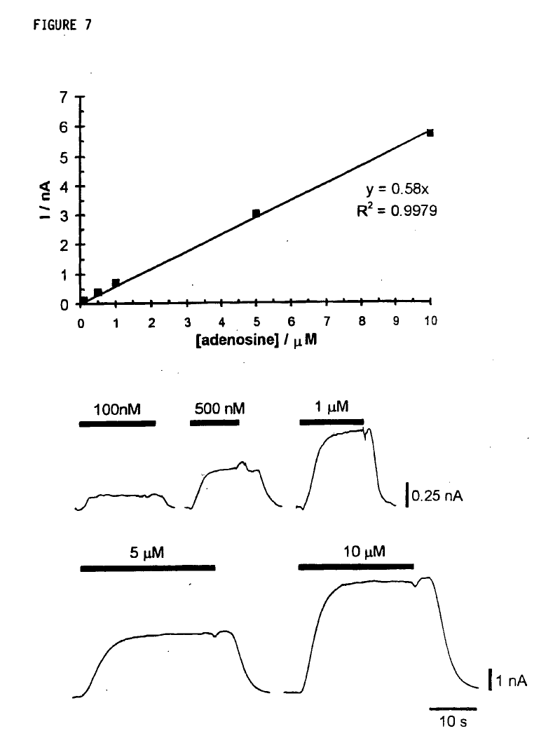

The sensor responded to concentrations as low as 100 nM and presented a linear response up to 20 mM. We have made sensors that range in surface area from 0.12 to 2.3 mm² (with diameters from 25 to 100 mm and lengths from 0.3 to 2 mm). The specific sensitivity per unit area of these sensors did not depend on the size and ranged from at least 100 mA M⁻¹cm² to 222 mA M⁻¹cm² with careful surface preparation (FIG. 7). The standard deviation of the noise for a typical sensor such as that in FIG. 7 is about 6 pA. A signal that is more than 2 times this magnitude will be readily detectable, giving a lower limit of 12 nM adenosine for the sensor illustrated in FIG. 7. Like Cosnier (Cosnier, et al., 1998) we found that an initial layer of the lactobionic derivative polymer greatly enhanced the sensor performance, presumably because it provides a more hydrophilic environment for the enzymes. However, unlike Cosnier it was found that a third layer of the lactobionic polymer on top of the enzyme layer severely reduced sensor performance by causing the responses to adenosine, inosine and xanthine to fade rapidly with time. This effect appeared as if it were product inhibition and may be caused by the trapping of products within the matrix by the additional lactobionic polymer layer.

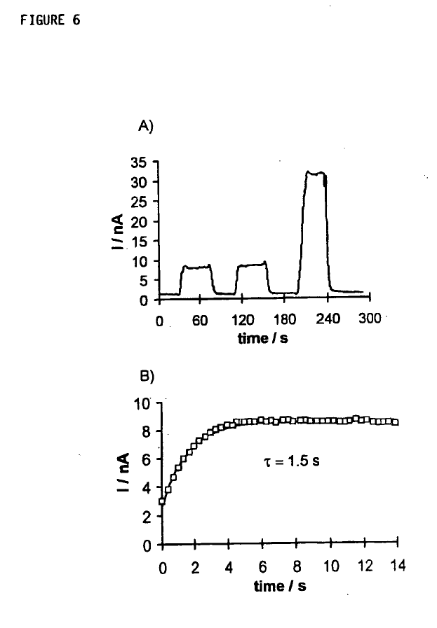

The sensors responded quickly to changes in adenosine concentrations and had a 10-90% rise time that ranged from 1 to 3.3 s with a mean value of 2±0.23 s (n=10, FIG. 6B). This is considerably better than the mark-1 adenosine sensor, shown in WO 00/07877, which required 1-2 mins to reach full response. The sensor maintained its initial sensitivity for at least two weeks if stored at 4° C. in phosphate buffer; nevertheless, after being used in vivo, some loss of sensitivity was seen. This ranged from very occasionail total loss to no loss. On average the sensors lost 57% of their sensitivity after being used in vivo (mean value from 15 sensors). This could be caused by physical damage of the polymer resulting from the mechanics of contact with tissue; loss of enzymatic activity due to proteases released from the tissue, or poisoning of the electrode by adsorbed proteins. We favour the release of proteases from tissue as the extent of loss also depended on the nature of the tissue—e.g. use with brain slices entailed significantly less loss of sensitivity compared to, the spinal cord (E. Llaudet, B. Frenguelli & N. Dale unpublished observations).

The addition of CH₃CN to the monomer-enzyme mixture greatly improved the probe sensitivity. This might be due to better solubilization of the monomers. Alternatively, some strong interactions between the enzymes' external residues and the amphiphilic monomer might have been weakened by the acetonitrile molecules. This would potentially improve the tertiary structure of the enzymes within the polymeric matrix leading to higher catalytic activity. Inclusion of CH₃CN could also allow the formation of a more ordered polymer, there is evidence that polypyrrole will swell in acetonitrile (Zhou et al., 1996). Either of these mechanisms would allow better diffusion of the substrates and products to and from the enzymes and thus improve sensor performance. The sensitivity of the sensor can be greatly increased by depositing extra layers of each enzyme; the multilayer configuration can almost double the sensor signal but on the other hand sensor response times can increase too. An appropriate strategy can thus be adopted depending on whether speed or sensitivity of analyte detection is most desirable.

Use In Vivo

Our previous work with an earlier design of sensor has demonstrated how extracellular adenosine levels rise during swimming episodes. This increase in adenosine causes the slowing (manifest by a lengthening of cycle period) and ultimately the termination of motor activity in Xenopus embryos (Dale, 1998; Dale and Gilday, 1996; Brown and Dale, 2000). We therefore tested whether the new sensor could also measure adenosine release from the spinal cord and whether the much smaller dimensions of the new sensor would permit spatial localization of the sources of adenosine.

One advantage of using electropolymerized coatings to entrap enzymes is the possibility of uniformly coating irregular surfaces. This permits great flexibility in the shape of the sensing element which can be made in a manner most appropriate to the morphology of the target tissue. In the present study a thin and long structure was needed to record along the rostro-caudal axis of the spinal cord of Xenopus embryo. The probe had to be thin enough to differentiate between dorsal and ventral parts of the cord, where different cells are located and, at the same time, long enough to encompass most of the length of the cord, as adenosine release is believed to occur from the whole population of motor pattern generation cells, which are evenly distributed along the cord axis.

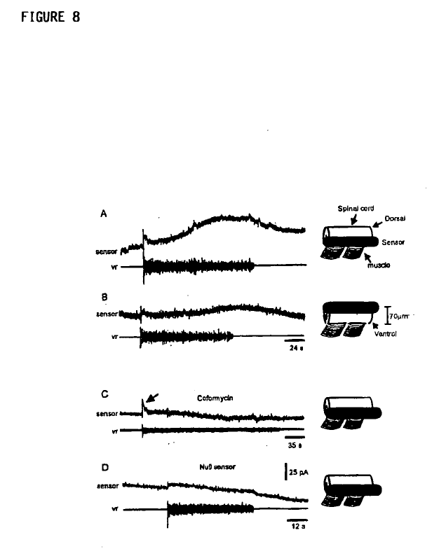

The new sensor clearly recorded a current that developed slowly during swimming motor activity (FIG. 8A) and returned to baseline following the end of activity. To test whether the recorded signal was produced by adenosine, a blocker of adenosine deaminase, coformycin, was applied. This makes the sensor insensitive to adenosine but still able to detect inosine, xanthine and any other nonspecific electroactive interferents that may be present. This gives a convenient test of the origin of the sensor signal that can be applied in every experimental context. As shown in FIG. 8C the slow rise in sensor current during swimming activity disappears when coformycin was applied. A fast, initial rise still occurs in the presence of coformycin suggesting that initial transient is not due to adenosine release. When a null sensor (no enzymes in polymer matrix) was used only a small baseline shift was seen demonstrating that the response to nonspecific interferents was very small in this experimental context (FIG. 8D). Although the sensor potential is held at 500 mV and many species could be directly oxidized at its surface, the fact that only a very small current was recorded when no enzymes were present in the null probe suggests that the polymer layers may act as partial barriers to electroactive species. We have found that the null sensors are half as sensitive to ascorbate than bare Pt without the polymer coating suggesting a weak barrier function.

The initial transient increase in sensor current (too fast to be resolved with the original mark-1 sensor recordings (Dale, 1998)) was not blocked by coformycin was thus not due to release of adenosine. Nevertheless this transient signal was not seen with null sensors and therefore depended upon the presence of the detecting enzymes. By making recordings with sensors containing only xanthine oxidase we have accumulated evidence that this transient signal is due to the fast release of xanthine—a mechanism previously undescribed in the nervous system (E. Llaudet & N. Dale, unpublished observations).

To test the capabilities of the new sensor to localize spatial release of adenosine we investigated whether we could detect differences in release between the ventral and dorsal regions of the spinal cord. When placed ventrally in gentle contact with the spinal cord the sensor recorded a large signal (FIG. 8A). However when contacting the surface of the spinal cord more dorsally (FIG. 8B) the signal was smaller and significantly delayed. This suggests that adenosine is produced in the very ventral part of the cord and that the ectonucleotidases responsible for its production must also be localized ventrally. Note that the diameter of the spinal cord is around 70 mm, and is smaller than the dimensions of the mark-1 sensor. We have therefore been able to demonstrate differential production of adenosine at a level of spatial resolution smaller than the dimensions of the previous sensor.

The superior temporal resolution of the new sensor also allowed us to quantify how the change in adenosine concentration throughout motor activity relates to its biological effect—the lengthening of motor cycle period (Dale, 1998; Dale and Gilday, 1996; Brown and Dale, 2000). In FIG. 9 the cycle period of a swimming episode (top trace) has been plotted on the same time axis as the simultaneously recorded sensor signal (bottom trace). Swimming was evoked by a stimulus to skin (at 10 s) and the rapid increase of cycle period over the first few seconds of activity reflects the decay of this sensory input. As swimming proceeds a slower increase in cycle period is seen. This reflects an intrinsic nin-down of motor activity, which is controlled by adenosine (Dale, 1998; Dale and Gilday, 1996). The recording shows that the slow increase of adenosine levels precedes this change. A plot of cycle period against sensor current (FIG. 6, inset) demonstrates a linear correlation between the cycle period and change in sensor current. The mean gradient of the regression line in 6 experiments showed that adenosine had to increase by 21±9.8 am to effect a 1 ms lengthening of cycle period.

Sensor geometry (length, diameter) can easily be modified for other experimental conditions such as recording from brain slices in vitro, or for implantation into deeper nuclei in the nervous system in vivo without causing significant damage. In comparison to the damage wrought by implantation of a mark-1 sensor (250 mm diameter), the new sensors of 25 mm diameter will destroy a volume of tissue when implanted into the brain that is 100-fold smaller. This great reduction in tissue damage and trauma will make the subsequent determinations much more physiologically meaningful. Indeed in recent experiments we have used the biosensor to successfully localize adenosine release to a small portion of the nucleus tractus solitarus of adult rats, buried between 300 and 800 mm beneath the brain surface (E. Llaudet, A. Gourine, N. Dale & K. M. Spyer, unpublished observations). This suggests that the polymer is robust enough to be inserted into tissue without being scraped off.

### CONCLUSIONS

The new sensor represents a considerable advance over the previous design. Firstly it is much smaller. The diameters of the smallest sensors we have made to date (25 mm) approach the dimensions of many neurons within the brain. The smaller size of the new sensor permits the accurate localization of adenosine release with a resolution of 10's of mm. The new sensors respond much faster to changes in analyte concentration, which gives the possibility of detecting fast release events and determining more precisely the temporal relationships between purine release and their biological actions. Thirdly the new sensors exhibit considerably higher sensitivity—exceeding 200 mA M⁻¹cm² as compared to around 30-50 mA M⁻¹cm⁻² for the mark-1 sensor.

### REFERENCES

Bartlett, P. N., Cooper, J. M., 1993. A review of the immobilization of enzymes in electropolymerized films. J. Electroanal. Chem., 362, 1-12.
Brown, P. E., Dale, N., 2000. Adenosine A(1) receptors modulate high voltage-activated Ca2+ currents and motor pattern generation in the Xenopus embryo. J. Physiol. 525, 655-667
Brundege, J. M., Dunwiddie, T. V., 1997. Role of adenosine as a modulator of synaptic activity in the central nervous system. Adv. Pharmacol. 39, 353-391.
Cass, A. E. G., 1990. Biosensors A Practical Approach, Oxford University Press.
Clark, Jr. L. C., Lyons, C., 1962. Electrode systems for continuous monitoring in cardiovascular surgery. Ann N.Y. Acad. Sci. 102, 29-45.
Coche-Guerente, L., Cosnier, S., Innocent, C., Mailley, P., 1995. Development of amperometric biosensors based on the immobilization of enzymes in polymer-films electrogenerated from a series of amphiphilic pyrrole derivatives. Anal. Chim. Acta 311, 23-30.
Cosnier, S., Poitry, S., Innocent, C., Allien, L., Tsacopoulos, M., 1997. An electrochemical method for making enzyme microsensors. Application to the detection of dopamine and glutamate. Anal. Chem. 69, 968-971.
Cosnier, S., Lepellec, A., Guidetti, B., Rico-Lattes, 1, 1998. Enhancement of biosensor sensitivity in aqueous and organic solvents using a combination of poly(pyrrole-ammonium) and poly(pyrrole-lactobionamide) films as host matrices. J. Electroanal. Chem 449, 165-171.
Dale, N., 1998. Delayed production of adenosine underlies temporal modulation of swimming in frog embryo. J. Physiol. 511, 265-272.
Dale, N., Gilday, D., 1996. Regulation of rhythmic movements by purinergic neurotransmitters in frog embryos. Nature 383, 259-263.
Dale, N., Pearson, T., Frenguelli, B. G., 2000, Direct measurement of adenosine release during hypoxia in the CA1 region of the rat hippocampal slice. J. Physiol. 526, 143-155.
Deronzier, A., Moutet, J-C., 1989. Functionalized polypyrroles—New molecular materials for electrocatalysis and related applications. Acc. Chem. Res. 22, 249-255.
McMillan, M. R., Burnstock, G., Haworth, S. G., 1999. Vasodilatation of intrapulmonary arteries to P2-receptor nucleotides in normal and pulmonary hypertensive newborn piglets. Brit. J. Pharmacol. 128, 543-548.
Pearson, T., Nuritova, F., Caldwell, D., Dale, N., Frenguelli, B. G., 2001. A depletable pool of adenosine in area CA1 of the rat hippocampus. J. Neurosci. 21, 2298-2307.
Pedata, F., Latini, S., Pugliese, A. M., Pepeu, G., 1993. Investigations into the adenosine outflow from hippocampal slices evoked by ischemia-like conditions. J. Neurochem. 61, 284-289.
Poitry, S., Poitry-Yamate, C., Innocent, C., Cosnier, S., Tsacopoulos, M., 1997. Detection of glutamate released by neurons with an enzyme-based microelectrode: applications and limitations. Electroch. Acta 42, 3217-3223.
Porkka-Heiskanen, T., 1999. Adenosine in sleep and wakefulness. Ann. Med. 31, 125-129.
Sawynok, J., 1998. Adenosine receptor activation and nociception. Eur. J. Pharmacol. 347, 1-19.
Slevin, C. J., Gray, N. J., Macpherson, J. V., Webb, M. A., Unwin, P. R., 1999. Fabrication and characterisation of nanometre-sized platinum electrodes for voltammetric analysis and imaging. Electrochem. Comm. 1, 282-288.
Zhou, M., Persin, M., Sarrazin, J., 1996. Methanol removal from organic mixtures by pervaporation using polypyrrole membranes. J. Membrane Sci. 117, 303-309.
Zimmermann, H., Braun, N., 1999. Ecto-nucleotidases—molecular structures, catalytic properties, and functional roles in the nervous system. Prog. Brain Res. 120, 371-385.

1. A biosensor comprising:
(i) a substrate comprising platinum or a platinum alloy;
(ii) a first layer formed on the substrate, the first layer comprising a sugar-derivative of a pyrrole; and
(iii) a second layer formed on the first layer, the second layer comprising an amphiphilic pyrrole and, within the second layer, one or more enzymes.

2. A biosensor according to claim 1, wherein the sugar-derivative of a pyrrole is a lactobionamide pyrrole.

3. A biosensor according to claim 1, wherein the amphiphilic pyrrole comprises a tertiary amine group.

4. A biosensor according to claim 1, wherein the substrate is platinum or a platinum-iridium alloy.

5. A biosensor according to claim 1 comprising two or more different enzymes within the second layer.

6. A biosensor according to claim 5, wherein each enzyme is deposited as a separate layer within the second layer, so that the second layer comprises two or more sub-layers of different enzymes.

7. A biosensor according to claim 6, comprising at least two layers of each enzyme.

8. A biosensor according to claim 1 comprising an oxidoreductase enzyme within the second layer.

9. A biosensor according to claim 8, wherein the oxidoreductase is xanthine oxidase.

10. A biosensor according to claim 9, additionally comprising nucleoside phosphorylase.

11. A biosensor according to claim 10, additionally comprising adenosine deaminase.

12. A biosensor according to claim 11, comprising a ratio adenosine deaminase: nucleoside phosphorylase: xanthine oxidase of approximately 1:1:5.

13. A biosensor according to claim 11, wherein the enzymes are deposited as separate sub-layers within the second layer, with xanthine oxidase deposited further away from the substrate than the nucleoside phosphorylase.

14. A biosensor according to claim 13, additionally comprising a layer of adenosine deaminase deposited closer to the substrate than the nucleoside phosphorylase.

15. A biosensor according to claim 13, comprising several layers of nucleoside phosphorylase and xanthine oxidase, and optionally adenosine deaminase.

16. A biosensor according to claim 1 additionally comprising a reference electrode.

17. A kit for detecting the presence and/or concentration of a substance comprising a biosensor according to claim 1.

18. A method of producing a biosensor according to claim 1, comprising the steps of:
(i) providing a substrate comprising platinum or a platinum alloy;
(ii) depositing a first layer comprising a sugar-derivative of a pyrrole; and
(iii) depositing a second layer, the second layer comprising an amphiphilic pyrrole and, within the second layer, one or more enzymes.

19. A method according to claim 18, wherein the second layer comprises two or more different enzymes, each enzyme being deposited in a solution comprising acetoniitrile as one or more separate sub-layers to form the second layer.

20. A method according to claim 18, wherein the first layer comprises a lactobionic pyrrole and is deposited in a solution comprising acetonitrile.

21-26. (canceled)

27. A method of detecting the presence and/or concentration of xanthine comprising use of the biosensor of claim 9.

28. A method according to claim 19, wherein the first layer comprises a lactobionic pyrrole and is deposited in a solution comprising acetonitrile.

29. A method of detecting xanthine or inosine comprising use of the biosensor of claim 11.

30. A method of detecting xanthine or inosine comprising use of the biosensor of claim 13.

31. A method of detecting one or more purines comprising use of the biosensor of claim 12.

32. A method of detecting one or more purines comprising use of the biosensor of claim 14.

33. A method of detecting the amount of a substance within a tissue, comprising exposing a biosensor according to claim 1 to a sample of tissue or body fluid in vivo or in vitro, and detecting an electrical current produced by the biosensor.

34. A method according to claim 33, wherein the tissue or body fluid is blood, brain, muscle, cardiac tissue, saliva or urine.

35. A method according to claim 1, wherein the substance is adenosine.

36. A biosensor according to claim 14 comprising several layers of nucleoside phosphorylase and xanthien oxidase, and optionally adenosine deaminase.
